(19) United States
(12) Patent Application Publication
(10) Pub. No.: US 2022/0018860 A1
(43) Pub. Date: Jan. 20, 2022

(54) SAMPLE PREPARATION AND TEST METHODS FOR DIAGNOSING CHRONIC WASTING DISEASE

(71) Applicant: Davin Henderson, Fort Collins, CO (US)

(72) Inventor: Davin Henderson, Fort Collins, CO (US)

(21) Appl. No.: 17/378,009

(22) Filed: Jul. 16, 2021

Related U.S. Application Data
(60) Provisional application No. 63/052,721, filed on Jul. 16, 2020.

(51) Int. Cl. G01N 33/68; G01N 1/28

(52) U.S. Cl. CPC G01N 33/6896; G01N 2001/2866; G01N 1/286

(57) ABSTRACT

A prion disease test kit is provided that allows for quick sample collection by an untrained individual and accurate testing of the sample in a lab, which is remote from the sample collection location. The prion disease test kit allows a lay person to easily collect tissue samples from the field and submit the collected tissue samples for prion disease testing, such as testing for chronic wasting disease, at a different location.

### RELATED APPLICATIONS

This application claims the priority benefit under 35 U.S.C. §119(e) of U.S. Provisional Patent Application Ser. No. 63/052,721 entitled “SAMPLE PREPARATION AND TEST METHODS FOR DIAGNOSING CHRONIC WASTING DISEASE,” filed Jul. 16, 2020, the entire disclosure of which is incorporated herein by reference.

### BACKGROUND

### 1. Field of the Invention

The present disclosure generally relates to the diagnosis of prion-related diseases.

### 2. Description of the Related Art

Generally, prion diseases are fatal neurodegenerative disorders that are caused and spread via abnormal prion proteins. An example of a prion disease is chronic wasting disease (“CWD”). The infectious agent, or prion, of prion diseases appears to be composed primarily of an abnormal, misfolded, oligomeric form of a protein (i.e., a “prion”), which are formed post-translationally from normal cellular proteins. These abnormal prions, which in purified form can resemble amyloid fibrils, may induce the polymerization and conformational conversion of normal cellular proteins to infectious abnormal prion proteins. Thus, these abnormal prions may self-propagate in the form of seeded or templated polymerization.

CWD is a fatal contagious prion disease generally affecting cervid species (e.g., deer, elk, and moose), which is characterized by neurodegeneration, emaciation, and abnormal behaviors. CWD is the only known prion disease to spread horizontally through wild populations, in which it continues to expand in prevalence and range in North America. As a prion disease, CWD is caused by a pathogenic, misfolded conformation of the normal, natively folded cellular protein to a pathogenic prion conformer (variously designated PrP^{CWD}, PRP^{SC}, or PrP^{D}).

Natural infection and transmission of CWD likely occurs through oral and nasal mucosal contact with infectious prions. The disease generally begins when the infectious prion induces continuous misfolding of the normal cellular proteins into a disease-associated, protease-resistant form, which aggregates into amyloid fibrils. In several prion diseases, including CWD, prions accumulate first in the systemic lymphoid tissues before entering the central nervous system.

The diagnosis of CWD has conventionally relied on the demonstration of the disease associated misfolded CWD prion protein in the brain or retropharyngeal lymph node tissue by immunodetection methods, such as ELISA and immunohistochemistry (IHC). However, the success of these methods generally relies on a quality sample of tissues, which requires both anatomical knowledge and considerable dissection to collect.

### SUMMARY

One or more embodiments of the present invention provide a prion disease test method and/or kit that allows for quick sample collection by an untrained individual and accurate testing of the sample in a lab that is remote from the sample collection location.

One or more embodiments of the present invention generally concern a method for testing for a prion disease. Generally, the method comprises: (a) receiving a third eyelid sample extracted from a mammalian subject; (b) subjecting at least 50 weight percent of the third eyelid sample to homogenization to thereby form a homogenized eyelid sample; and (c) testing the homogenized third eyelid sample for a prion disease.

One or more embodiments of the present invention generally concern a method for testing for chronic wasting disease. Generally, the method comprises: (a) receiving a third eyelid sample extracted from an ungulate; (b) subjecting at least 50 weight percent of the third eyelid sample to homogenization to thereby form a homogenized eyelid sample; and (c) testing the homogenized eyelid sample for chronic wasting disease.

### BRIEF DESCRIPTION OF THE FIGURES

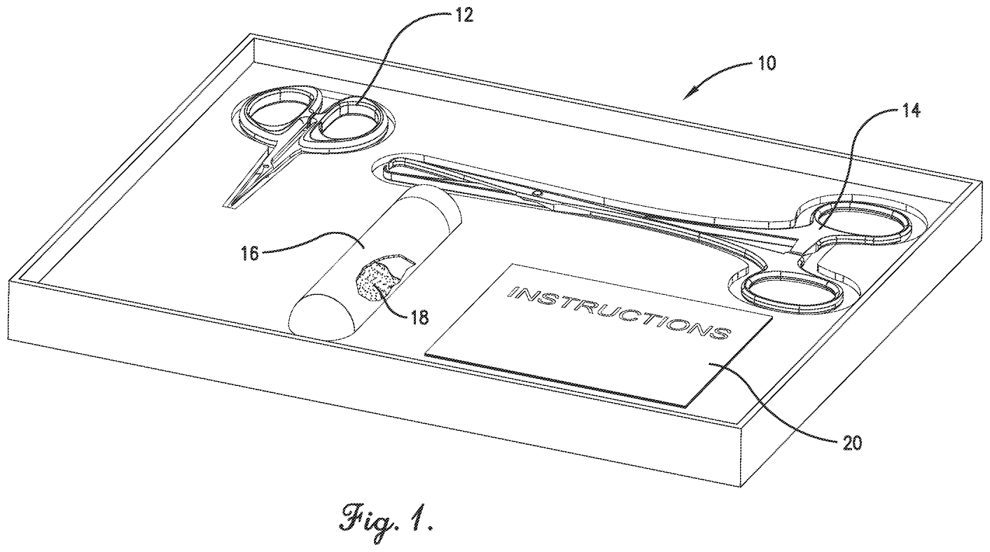

Embodiments of the present invention are described herein with reference to the following drawing FIGURE (FIG. 1), which depicts an exemplary tissue sample collection kit according to one embodiment of the present invention.

### DETAILED DESCRIPTION

In order to address the deficiencies of previous CWD diagnostic methods, it has been discovered that the use of third eyelids from CWD-infected cervids may be easily extracted by any lay person and submitted for CWD testing by using real-time quaking induced conversion (“RT-QuIC”). The RT-QuIC test combines features of quaking-induced conversion (QuIC) and amyloid seeding assay (ASA) methods and involves prion-seeded conversion of the alpha helix-rich form of bacterially-expressed recombinant cellular protein to a beta sheet-rich amyloid fibrillar form. The RT-QuIC test is as sensitive as an animal bioassay but can be accomplished in two days or less.

More particularly, the present disclosure is generally directed to kits and methods for collecting samples from mammalian subjects and diagnosing such samples for CWD. For example, this includes kits that allow any individual, including a lay person, to extract a tissue sample from a mammalian subject out in the field for subsequent prion disease testing, including CWD testing. Furthermore, such kits may contain sample containers that include optimal reaction reagents to perform a CWD RT-QuIC test. Thus, a lay person may collect the tissue sample by themselves in the field, place a collected tissue sample in the sample container containing all of the necessary RT-QuIC reagents, and then submit the sealed container to a prion disease (e.g., CWD) testing facility.

Exemplary kits and their contents are discussed below in greater detail. It should be noted that all of the following kit components are not mutually exclusive (unless otherwise noted) and, therefore, may be combined in any manner as so desired.

In one or more embodiments, a kit is provided for extracting a tissue sample from a mammalian subject for prion disease testing. As shown in FIG. 1, the exemplary kit 10 may comprise: (a) an extraction tool 12 for removing the tissue sample from the mammalian subject; (b) a grasping tool 14 for handling the tissue sample; (c) a sealable tissue sample container 16 for storing an extracted tissue sample; (d) at least one sample preservation or preparation material 18; and (e) instruction information 20 regarding extraction of the tissue sample, handling of the tissue sample, and/or handling of the sample container.

In one or more embodiments, a method for producing a kit for extracting a tissue sample from a mammalian subject for prion disease testing is provided. Generally, the method for producing such a kit may comprise: (a) providing an extraction tool for removing the tissue sample from the mammalian subject; (b) providing a grasping tool for handling the tissue sample; (c) providing a sealable tissue sample container for storing an extracted tissue sample; (d) providing at least one sample preservation or preparation material; (e) providing instruction information regarding extraction of the tissue sample, handling of the tissue sample, and/or handling of the sample container; and (f) combining the extraction tool, the grasping tool, the tissue sample container, the sample preservation or preparation material, and the instruction information in a single package to thereby form the kit.

It should be noted that the kit can be in the form of a single package (e.g., a box, a container, a tube, etc.) that contains all of components of the kit, as shown in FIG. 1. Thus, in such embodiments, the kit 10 may be in the form of a single package containing the extraction tool 12, the grasping tool 14, the sealable tissue sample container 16, the sample preservation or preparation material 18, and the instruction information 20. In various embodiments, the single package of the kit can be in the form of a resealable packaging that can be resealed with the collected tissue sample and sent to a prion testing facility, such as a CWD testing facility.

The focus of the kits of the present disclosure is to permit a lay person, such as a hunter, to easily extract a tissue sample from their harvested prey at any location, including the field. Thus, a lay person can obtain a kit, such as purchasing the kit at a retail store and/or on an online store and use the kit to easily extract the desired tissue sample from their harvested prey while in the field.

As noted above, the kits may comprise an extraction tool for extracting the desired tissue sample from the mammalian subject. The extraction tool may comprise scissors and/or a scalpel. In certain embodiments, the extraction tool may comprise a disposable extraction tool, such as disposable scissors.

As noted above, the kits may comprise a grasping tool for handling the desired tissue sample from the mammalian subject. Typically, the grasping tool may comprise forceps. In certain embodiments, the grasping tool may comprise a disposable grasping tool, such as disposable forceps.

As noted above, the kits may comprise a sealable tissue sample container for storing an extracted tissue sample. Exemplary sealable tissue sample containers can include tubes with an attached lid and/or tubes with a lid that may be screwed on. Thus, the extracted tissue may be placed into this sample container and the container may then be sealed to deter outside contamination. Generally, the sample container may comprise a tube with a volume of at least 0.1, 0.5, 1, 2, 3, 4, 5, 6, 7, 8, 9, or 10 mL and/or not more than 200, 150, 100, 50, 25, or 10 milliliters (mL). Furthermore, the tubes may be made from a plastic that will not affect the extracted tissue sample stored inside of it. For instance, the tubes may be made of polypropylene and/or polyethylene.

As noted above, the kits may comprise instruction information regarding extraction of the tissue sample, handling of the tissue sample, and/or handling of the sample container. Such information may also include instructions for mailing or dropping off the collected tissue sample to a prion disease testing facility, such as a CWD testing facility. In one or more embodiments, the instruction information may comprise physical instructions, a text link to online instructions, and/or a QR code that links to online instructions. For example, the instruction information may include a QR code that, when scanned by a smart phone device, leads the individual to an online video and/or instruction website for using the extraction kit.

Additionally, in various embodiments, the kits may comprise a mailing container for packaging the extracted tissue sample and sending it off to a prion disease testing facility, such as a CWD testing facility. The mailing container may comprise a sealable box or envelope, which may be sealed and shipped to the testing facility. In certain embodiments, the mailing container may comprise thermal packaging so as to maintain the extracted tissue sample at a desired temperature while in transit.

As noted above, the kits may comprise at least one sample preservation and/or preparation material. Generally, such materials may be packaged separately from the sealable sample container and/or preloaded into the sample container. In one or more embodiments, the sample preservation and the preparation material in the kit comprises a preservative for the tissue sample, a prion protein amplification substrate, a dilution liquid, a plurality of tissue homogenization beads, an RT-QuIC reaction buffer, or combinations thereof.

In certain embodiments, the kit comprises a plurality of tissue homogenization beads preloaded in the sample container. The homogenization beads can be subsequently used at the prion disease testing facility to homogenize the extracted tissue sample within the sample container. In other words, due to the presence of the homogenization beads in the sample container, the extracted tissue sample may be subsequently homogenized directly in the sample container. Generally, the homogenization beads may comprise ceramic beads (e.g., zirconium oxide beads), glass beads (e.g., silica beads), stainless steel beads, corundum beads, or combinations thereof.

In certain embodiments, the kit comprises a dilution liquid, which may be preloaded in the sample container. The dilution liquid may comprise a phosphate buffered saline (PBS) and/or one or more detergents. In one or more embodiments, the dilution liquid may comprise at least 0.001, 0.005, 0.01, 0.05, 0.1, or 0.5 weight percent and/or not more than 20, 15, 10, 9, 8, 7, 6, 5, 4, 3, 2, or 1 weight percent of one or more detergents, based on the total weight of the dilution liquid. Exemplary detergents include anionic detergents and/or nonionic detergents, such as sodium dodecyl sulphate (SDS).

In certain embodiments, the kit comprises an RT-QuIC reaction buffer, which may be preloaded in the sample container. Generally, the RT-QuIC reaction buffer may comprise NaH₂PO₄, NaCl, EDTA, thioflavin T, or combinations thereof.

In certain embodiments, the kit comprises a prion protein amplification substrate, which can, optionally, be preloaded into the sample container and subsequently used for the amplification of the test prion for RT-QuIC testing. When it is required for the substrate to be maintained in a frozen state, it may be impractical to include the substrate as part of the kit. However, certain substrates may not require freezing or refrigeration and may readily be included in the kit. The protein substrate may be from the same species as the source of the sample or may be from a different species relative to the sample source. Exemplary sources of the protein substrate can include bovine, ovine, hamster, rat, mouse, canine, feline, cervid, human, or non-human primate protein substrates.

Exemplary detergents, prion protein amplification substrates, dilution liquids, and preservatives are disclosed in U.S. Pat. No. 8,216,788 and U.S. Patent Application Publication No. 2006/0263767, the disclosures of which are incorporated herein by reference in their entireties.

The kits described herein may be purchased at a retail store and/or on an online store and used by any individual, including a lay person.

As noted above, the kits may be used to collect a tissue sample from a mammalian subject. Generally, in various embodiments, the mammalian subject comprises an ungulate, such as an animal that is a ruminant. In certain embodiments, the mammalian subject is an animal from the Cervidae family, such as white-tailed deer, mule deer, red deer, sika deer, axis deer, elk, moose, caribou, or reindeer.

In certain embodiments, the mammalian subject comprises a harvested wild mammalian subject, such as a harvested wild ungulate. As used herein, the term “harvested wild mammalian subject” and “harvested wild ungulate” refer to wild mammalian subjects and ungulates that are not domesticated livestock and were harvested as the result of a hunting endeavor. Thus, a harvested wild mammalian subject and a harvested wild ungulate would include animals that were harvested as a result of a hunt and not encompass animals that are raised and harvested in a domesticated setting (e.g., a livestock ranch, but not including hunting ranches).

Generally, the tissue sample collected using the kit may comprise a variety of tissue samples. In one or more embodiments, the extracted tissue sample can comprise rectoanal mucosa-associated lymphoid tissue (RAMALT), spleen tissue, tonsil tissue, skin tissue, and/or eyelid tissue. In certain embodiments, the extracted tissue sample may come from a third eyelid of a ruminant animal, particularly an animal from the Cervidae family.

The third eyelid is a nictitating membrane found in many animal species located between the globe of the eye and the lower eyelid, thereby making it easily accessible without special anatomical training. In ruminants, including cervids, the membrane contains lymphoid tissue organized into the lymphoid follicles with germinal centers where prion protein can accumulate at early stages of disease.

It has been observed that third eyelid tissue can be used to detect CWD in cervids, such as deer and elk, including those in pre-symptomatic stages of infection. More particularly, it has been observed that the third eyelid can be used in an RT-QuIC assay to consistently detect amyloid seeding activity from a variety of CWD-infected, symptomatic subjects with little false positivity. As the third eyelid is an easily accessible tissue, it has potential to aid in surveillance and screening programs.

Due to the instructions provided in the kits, any lay person, such as a hunter who is harvesting their prey or an employee at a meat processor, may utilize the kits to extract the desired tissue samples from the mammalian subjects. In other words, the kits can be easily used by any lay person who is not formally trained in extracting tissue samples from animals. As used herein, a “lay person not formally trained in extracting test samples from animals” excludes an individual who: (1) has a post-secondary degree, such as an bachelor's degree, a master's degree, or a doctorate, related to the biological sciences, such as, for example, zoology, anatomy, and epidemiology and/or (2) has previously been employed as and/or is currently employed as a lab technician in a laboratory focused on one or more biological applications. Thus, using a kit described herein, a lay person not formally trained in extracting test samples from animals may extract one or more of the above-referenced tissue samples from a mammalian subject and submit the extracted tissue sample for further testing at a prion disease testing facility. An exemplary lay person may include a hunter, a non-medical entity, or a non-veterinary entity.

In various embodiments, an individual lay person may extract a third eyelid sample from a mammalian subject, such as the third eyelid from an animal from the Cervidae family, at a first location. This first location can be a remote location that is not a laboratory setting or a prion disease testing facility. For example, the first location can be a remote, undeveloped location where the subject was harvested. Subsequently, the individual may submit the extracted third eyelid sample (optionally with a mailing container from the kit) via mail and/or courier to a prion disease testing facility at a second location, which is separated from the first location by at least 1, 5, 10, 15, 20, 25, 30, 35, 40, 45, or 50 miles. This prion disease testing facility may comprise the chronic wasting disease test facility, particularly an RT-QuIC test facility. After testing the extracted sample, the prion disease testing facility may then send (e.g., via mail or email) the results back to the individual, a government entity, and/or a regulating entity.

Due to the kits described herein, a lay person, such as a hunter, may be able to easily extract a tissue sample for subsequent CWD testing from a harvested wild cervid while in the field.

As discussed above, the extracted tissue sample may be submitted to a prion disease testing facility, particularly a CWD testing facility for further testing. While at the testing facility, the extracted sample may be at least partially homogenized and subjected to RT-QuIC testing.

In various embodiments, the extracted tissue sample comprises a third eyelid extracted from a cervid. It has been demonstrated that RT-QuIC is the only test sensitive enough to be able to avoid having to dissect out a sub-sample of the extracted third eyelid, thereby saving a lot of time and labor, especially when testing on a commercial scale. Consequently, the entire third eyelid sample may be homogenized for subsequent RT-QuIC testing.

In one or more embodiments, at least 50 55, 60, 65, 70, 75, 80, 85, 90, 95, or 99 weight percent of the extracted third eyelid sample may be subjected to homogenization prior to RT-QUiC testing. In certain embodiments, the entire third eyelid sample is subjected to homogenization. In such embodiments, the extracted eyelid sample may comprise at least 20, 25, 30, 35, 40, 45, 50, 55, 60, 65, 70, 75, 80, 85, 90, 95, or 99 percent of a third eyelid of the mammalian subject.

In various embodiments, the homogenization process involves: (i) adding homogenization beads and a dilution liquid to the third eyelid sample to form a pre-homogenized mixture and (ii) agitating the pre-homogenized mixture to thereby form the homogenized eyelid sample. Alternatively, in certain embodiments, homogenization may be carried out using a mortar and pestle to homogenize the tissue sample. In certain circumstances, when the above-referenced kits are used and the sample containers are preloaded with homogenization beads, the homogenization step may occur in the sample container in which the extracted eyelid sample is stored. In such embodiments, the homogenization step may be carried out using the homogenization beads and dilution liquid discussed above. Furthermore, any conventional homogenization device may be used, such as a Bead Ruptor 24 (Omni International) or a Mini Beadbeater (Biospec Products).

After homogenization, the homogenized sample may be subjected to a separation step (e.g., in a centrifuge) and then subjected a prion disease test, particularly an RT-QuIC test for CWD.

The RT-QuIC assay can rapidly determine relative prion concentrations with a sensitivity that rivals that of animal bioassays, but with greatly reduced time and cost. Furthermore, it has been demonstrated that RT-QuIC can enhance CWD detection in tissue samples in which prion concentrations are quite low. The ability to detect prions rapidly and sensitively is an important asset in managing CWD. Early prion detection in individuals is critical to the prevention of spread and the initiation of potential treatments.

Exemplary RT-QuIC testing procedures are described in U.S. Pat. No. 8,216,788; “Rapid End-Point Quantitation of Prion Seeding Activity with Sensitivity Comparable to Bioassay” by Wilham et al.; “Rapid Antemortem Detection of CWD Prions in Deer Saliva” by Henderson et al.; and “Longitudinal Detection of Prion Shedding in Saliva and Urine by Chronic Wasting Disease-Infected Deer by Real-Time Quaking-Induced Conversion” by Henderson et al.; the disclosures of which are incorporated herein by reference in their entireties.

RT-QuIC generally offers the potential for sensitive ante-mortem detection of prions in a single round assay. In RT-QuIC, a prion seed converts recombinant normal prion protein into amyloid fibrils, an event detectable by the binding of thioflavin T (ThT). Alternating cycles of incubation and shaking are used to facilitate fibril fragmentation and re-seeding, thus amplifying minute amounts of prion seed to a detectable level. Previous studies have validated RT-QuIC for the identification of CWD prions in brain, lymph nodes, and other tissues, as well as in secretions and excretions.

A number of the conditions and components of the RT-QuIC may be modified as necessary. These modifiable conditions can include the concentration of detergent in the dilution liquid, the type of detergent in the dilution liquid, the amount of dilution liquid added to the homogenized tissue sample, the pH of the reaction mixture, the shaking speed during the shaking cycles, the reaction temperature, and the duration of the shaking/rest cycles (which are generally about 1:1 in duration).

In various embodiments, the RT-QuIC may comprise a number of cycles, with each cycle comprising alternating sessions of shaking and rest. Generally, in various embodiments, the RT-QuIC test may comprise at least 25, 50, 75, 100, 125, 150, 175, 200, 225, or 250 cycles. Additionally, the duration of each cycle may be varied as necessary and the cycle time may be influenced by the temperature of the reactions. For example, each cycle may occur for at least 1, 2, 3, 4, 5, 6, 7, 8, 9, 10, 11, 12, 13, 14, or 15 minutes and/or less than 60, 45, 30, 25, 20, 15, or 10 minutes. Furthermore, in various embodiments, the shaking and resting sessions may be alternated at approximately equal durations in length. For example, each shaking and rest session may alternate and last for at least 0.5, 1, 2, 3, or 4 minutes and/or less than 15, 14, 13, 12, 11, 10, 9, 8, 7, 6, 5, 4, 3, 2, or 1 minutes.

Generally, RT-QuIC may be performed by first diluting the homogenized tissue sample with the dilution liquid described herein at a liquid to sample ratio of at least 5:1, 6:1, 7:1, 8:1, or 9:1 and/or less than 20:1, 15:1, or 12:1. Generally, in certain embodiments, the tissue homogenates may be diluted at about a 10:1 ratio in a SDS/PBS dilution liquid. Afterwards, the diluted sample may then be loaded into a well of a well plate. Typically, the well plate may comprise a 96-well plate, a 384-well plate, or a 1536-well plate. Each well may also contain or be preloaded with at least 0.1 mg/ml of a prion protein substrate (e.g., 90-231 Syrian hamster recombinant prion protein) and a RT-QuIC reaction buffer (e.g., 20 mM NaH₂PO₄, 320 mM NaCl, 1.0 mM EDTA, and 1 mM thioflavin T).

In certain embodiments, each RT-QuIC test may comprise around 250 cycles and lasts around 62.5 hours. Generally, each cycle may comprise alternating cycles of shaking and rest, with each shaking and rest cycle being about the same length. For example, each RT-QuIC cycle may be 15 minutes in duration and comprise alternating sessions of one minute of shaking and one minute of rest in a BMG Labtech Fluostar™ fluorometer/plate reader.

Fluorescence readings during the RT-QuIC test may be recorded by the reader at the end of each cycle with an excitation of 450 nm and emission of 480 nm, using a gain of 1,700.

Generally, in various embodiments, the RT-QuIC test may be carried out at a temperature of at least 30, 35, or 40° C. and/or less than 80, 75, 70, or 65° C. In certain embodiments, the RT-QuIC test may be carried out at a temperature ranging from 37 to 65° C.

Samples are deemed RT-QuIC positive if 100% of the replicates tested achieve the fluorescence threshold. Replicates from each sample are considered positive for amyloid formation if the fluorescence rose above the threshold of 5 standard deviations above the average of initial baseline fluorescence readings. Statistical analysis of data may be performed using GraphPad Prism software.

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “having,” “has,” and “have” have the same open-ended meaning as “comprising,” “comprises,” and “comprise” provided above.

As used herein, the terms “including,” “include,” and “included” have the same open-ended meaning as “comprising,” “comprises,” and “comprise” provided above.

### Numerical Ranges

The present description uses numerical ranges to quantify certain parameters relating to the invention. It should be understood that when numerical ranges are provided, such ranges are to be construed as providing literal support for claim limitations that only recite the lower value of the range as well as claim limitations that only recite the upper value of the range. For example, a disclosed numerical range of 10 to 100 provides literal support for a claim reciting “greater than 10” (with no upper bounds) and a claim reciting “less than 100” (with no lower bounds).

Furthermore, terms leading a recited range containing a plurality of numerical values, such as “at least,” “not more than,” and “less than,” apply to all of the numerical values recited in the range listing. For example, “at least 1, 2, 3, or 4” should be interpreted as covering ranges of “at least 1, at least 2, at least 3, or at least 4.”

### Claims Not Limited to Disclosed Embodiments

The preferred forms of the invention described above are to be used as illustration only, and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

What is claimed is:

1. A method for testing for a prion disease, said method comprising:
(a) receiving a third eyelid sample extracted from a mammalian subject;
(b) subjecting at least 50 weight percent of said third eyelid sample to homogenization to thereby form a homogenized eyelid sample; and
(c) testing said homogenized third eyelid sample for a prion disease.

2. The method according to claim 1, wherein at least 90 weight percent of said third eyelid sample is subjected to homogenization.

3. The method according to claim 1, wherein said third eyelid sample is extracted by lay person not formally trained in extracting test samples from animals.

4. The method according to claim 1, wherein said mammalian subject comprises a harvested wild mammalian subject.

5. The method according to claim 1, wherein said testing comprises RT-QuIC testing.

6. The method according to claim 1, wherein said homogenization comprises: (i) adding homogenization beads and a dilution liquid to said third eyelid sample to form a pre-homogenized mixture and (ii) agitating the pre-homogenized mixture to thereby form said homogenized eyelid sample.

7. The method according to claim 1, wherein said mammalian subject comprises an ungulate mammal.

8. The method according to claim 1, wherein said third eyelid sample comprises a third eyelid of a ruminant.

9. The method according to claim 1, wherein said prion disease comprises chronic wasting disease.

10. The method according to claim 1, wherein said receiving comprises receiving said third eyelid sample in a mailing container.

11. A method for testing for chronic wasting disease, said method comprising:
(a) receiving a third eyelid sample extracted from an ungulate;
(b) subjecting at least 50 weight percent of said third eyelid sample to homogenization to thereby form a homogenized eyelid sample; and
(c) testing said homogenized eyelid sample for chronic wasting disease.

12. The method according to claim 11, wherein at least 90 weight percent of said third eyelid sample is subjected to homogenization.

13. The method according to claim 11, wherein said third eyelid sample is extracted by lay person not formally trained in extracting test samples from animals.

14. The method according to claim 11, wherein said ungulate comprises a harvested wild ungulate.

15. The method according to claim 11, wherein said testing comprises RT-QuIC testing.

16. The method according to claim 11, wherein said homogenization comprises: (i) adding homogenization beads and a dilution liquid to said third eyelid sample to form a pre-homogenized mixture and (ii) agitating the pre-homogenized mixture to thereby form said homogenized eyelid sample.

17. The method according to claim 11, wherein said ungulate comprises a ruminant.

18. The method according to claim 11, wherein said receiving comprises receiving said third eyelid sample in a mailing container.

19. The method according to claim 11, wherein said third eyelid sample in step (a) is contained in a sample container, wherein said sample container comprises a substrate, a dilution liquid, and an RT-QuIC reaction buffer.

20. The method according to claim 19, wherein said RT-QuIC reaction buffer comprises NaH₂PO₄, NaCl, EDTA, thioflavin T, or combinations thereof.
